(19) United States
(12) Patent Application Publication
(10) Pub. No.: US 2026/0026683 A1
(43) Pub. Date: Jan. 29, 2026

(54) VAGINAL SPECULUM

(71) Applicant: REVYN MEDICAL TECHNOLOGIES INC., Victoria (CA)

(72) Inventors: Keeley McCormick, Victoria (CA); Samantha Sperling, Esquimalt (CA); Joshua Latimer, Victoria (CA); Zoë Crookshank, Powell River (CA); Devon Carmichael, Victoria (CA)

(21) Appl. No.: 19/270,671

(22) Filed: Jul. 16, 2025

(30) Foreign Application Priority Data
Jul. 23, 2024 (CA) 3249343

(51) Int. Cl. A61B 1/32; A61B 1/303

(52) U.S. Cl. CPC A61B 1/32; A61B 1/303; A61B 2217/00

(57) ABSTRACT

Described are various embodiments of a vaginal speculum. One embodiment comprises a handle portion and a tubular insertion portion mounted at an angle to the handle portion at a manipulatable end thereof. The tubular insertion portion defines a line-of-sight channel therein from the manipulatable end to a resiliently collapsable insertion tip portion of the tubular insertion portion. The resiliently collapsable insertion tip portion is manufactured of a resiliently collapsable material of sufficient flexibility to collapse under manual pressure to reduce an insertion cross-section thereof for ease of insertion, while being of sufficient resiliency to expand and resist collapse from vaginal wall pressure once inserted. Some embodiments provide for camera and/or lighting features. Vaginal speculum kits are further described, optionally having a reusable handle portion and various single-use, disposable tubular insertion portions which are connectable thereto for different anatomies, examinations and procedures.

### CROSS REFERENCE TO RELATED APPLICATION

This application claims priority to Canadian Patent Application No. 3,249,343, filed Jul. 23, 2024, the entire disclosure of which is hereby incorporated herein by reference.

### FIELD OF THE DISCLOSURE

The present disclosure relates to medical devices, and, in particular, to a vaginal speculum and a vaginal speculum kit, wherein one or more components may be disposable.

### BACKGROUND

Vaginal speculums are medical devices or instruments used to widen the vaginal walls of a patient such that a medical practitioner can examine the vaginal canal and/or cervix of the patient. Vaginal speculums are employed in various gynecological procedures, including diagnostic procedures such as a Pap smear or test to test for cervical cancer in women.

Conventional vaginal speculums include two arms that meet at a hinge, forming a general duck bill configuration. The closed arms are inserted into the vagina and are opened using the hinge to widen the vaginal canal. A stop mechanism is typically included to hold the arms apart for the duration of the procedure, before being released to return the arms to the closed configuration. Such conventional vaginal speculums are typically manufactured of stainless steel to allow for sterilization between uses. More recently, plastic constructions intended for single use have become available in the market.

U.S. Pat. No. 4,046,140A teaches a Cervix Photographic Method in which a solid barrel-type speculum manufactured of a glass-like material such as Pyrex terminates in a 30 to 45 degree beveled end.

WO2018187235A1 teaches a Low-Profile Videoscopic Speculum with Working Port, which generally defines a barrel-type speculum made of a rigid material that terminates in a tapered section and in which an elongate insert having a wedge tip can be inserted such that the wedge tip and tapered section, in combination, allow for a more comfortable vaginal insertion of the speculum.

US20050277811 teaches a barrel-type Speculum having a lengthwise slot to provide space for surgical instruments and terminating in a bevelled insertion end having softened or rounded edges for ease of insertion.

U.S. Pat. No. 4,994,070 teaches an Apparatus for Dilating a Body Cavity and defines a vaginal speculum having a sheet member of inherently resilient material capable of being rolled into a narrow generally elongate shape, and released to expand under its inherent resilience to a dilated position in which the member forms a hollow, generally open-ended elongate shape having a substantially continuous sidewall.

This background information is provided to reveal information believed by the applicant to be of possible relevance. No admission is necessarily intended, nor should be construed, that any of the preceding information constitutes prior art or forms part of the general common knowledge in the relevant art.

### SUMMARY

The following presents a simplified summary of the general inventive concept(s) described herein to provide a basic understanding of some aspects of the disclosure. This summary is not an extensive overview of the disclosure. It is not intended to restrict key or critical elements of embodiments of the disclosure or to delineate their scope beyond that which is explicitly or implicitly described by the following description and claims.

A need exists for vaginal speculum that overcomes some of the drawbacks of known techniques, or at least, provides a useful alternative thereto. Some aspects of this disclosure provide examples of such speculums, in accordance with different embodiments.

In accordance with one aspect, there is provided a vaginal speculum, comprising: a handle portion; and a tubular insertion portion mounted at an angle to said handle portion at a manipulatable end thereof, and defining a line-of-sight channel therein from said manipulatable end to a resiliently collapsable insertion tip portion of the tubular insertion portion; wherein said resiliently collapsable insertion tip portion is manufactured of a resiliently collapsable material of sufficient flexibility to collapse under manual pressure to reduce an insertion cross-section thereof for ease of insertion, while being of sufficient resiliency to expand and resist collapse from vaginal wall pressure once inserted.

In one embodiment, the resiliently collapsable tip portion comprises a bevelled insertion edge formation.

In one embodiment, the bevelled insertion edge formation further comprises an axially extending lip at an axially utmost edge portion thereof.

In one embodiment, the beveled insertion edge formation is defined by a concave forward curving edge terminating in said axially extending lip.

In one embodiment, the resiliently collapsable tip portion is defined by a decreasing annular thickness.

In one embodiment, the tubular insertion portion comprises an outward bouquet portion towards the resiliently collapsable tip portion.

In one embodiment, the tubular insertion portion is integrally formed, with said resiliently collapsable tip portion, of said resiliently collapsable material.

In one embodiment, a collapsibility of said resiliently collapsable tip portion is greater than that of said manipulable end.

In one embodiment, the resiliently collapsable tip portion comprises a manually collapsible formation to facilitate resilient collapse. In one embodiment, the manually collapsible formation comprises a plurality of grooves running on any one or both of an interior surface or an exterior surface of the tubular insertion portion. In one embodiment, the resiliently collapsable material comprises any one or combination of: a medical grade silicone, a polysiloxane, an elastomer, or a biocompatible flexible resin.

In one embodiment, the tubular insertion portion is manufactured of any one of stainless steel, plastic, acrylic or a rigid silicone; and overlayed by any one of silicone, polysiloxane, elastomer or biocompatible flexible resin, to form said resiliently collapsable insertion tip portion extending axially therefrom.

In one embodiment, at least a portion of said tubular insertion portion is manufactured of one or more materials selected for transmission of light therethrough.

In one embodiment, the tubular insertion portion further comprises a coupler extending radially therefrom for coupling to said handle portion.

In one embodiment, the coupler comprises a disengageable coupling portion allowing for engagement and disengagement of said tubular insertion portion with said handle portion.

In one embodiment, the tubular insertion portion and said handle portion are mechanically coupled via a hinge mechanism to selectively adjust said angle.

In one embodiment, an unobstructive instrumentation groove is defined along an internal surface of said tubular insertion portion, or within an annular wall of said tubular insertion portion, to receive instrumentation therein for performing a medical examination or procedure.

In one embodiment, the grove comprises a snap-fit groove.

In one embodiment, the groove further comprises one or more securing members along a length thereof to secure said instrumentation therein.

In one embodiment, the groove is dimensioned to receive therein any one or both of: a lighting assembly or a camera assembly.

In one embodiment, the lighting assembly and/or said camera assembly are channeled from said grove to within said handle portion.

In one embodiment, the handle is ambidextrous.

In accordance with another aspect, there is provided a vaginal examination kit, comprising: a handle portion; and at least two exchangeable tubular insertion portions disengageably mountable at an angle to said handle portion at a manipulatable end thereof, and defining a line-of-sight channel therein from said manipulatable end to a resiliently collapsable insertion tip portion of the tubular insertion portion; wherein said resiliently collapsable insertion tip portion is manufactured of a resiliently collapsable material of sufficient flexibility to collapse under manual pressure to reduce an insertion cross-section thereof for ease of insertion, while being of sufficient resiliency to expand and resist collapse from vaginal wall pressure once inserted.

In one embodiment, each of said tubular insertion portions comprise a coupler extending radially therefrom allowing for engagement and disengagement of said tubular insertion portions with said handle portion. In one embodiment, each of the tubular insertion portions are single-use disposables.

Other aspects, features and/or advantages will become more apparent upon reading of the following non-restrictive description of specific embodiments thereof, given by way of example only with reference to the accompanying drawings.

### BRIEF DESCRIPTION OF THE FIGURES

Several embodiments of the present disclosure will be provided, by way of examples only, with reference to the appended drawings, wherein:

FIG. 1 is a front perspective view of a vaginal speculum, in accordance with one embodiment;

FIG. 2 is a rear perspective view of the vaginal speculum of FIG. 1;

FIG. 3 is a cross-sectional view of the vaginal speculum of FIG. 1, taken along the line A-A thereof;

FIG. 4 is a top plan view of the vaginal speculum of FIG. 1;

FIG. 5 is an enlarged front elevation view of an insertable portion of the vaginal speculum of FIG. 1;

FIG. 6 is an enlarged rear elevation view of a distal end of the vaginal speculum of FIG. 1;

FIG. 7 is a front perspective view of a two-part vaginal speculum, in accordance with another embodiment, having a shaft or insertion portion removable from a handle portion;

FIG. 8 is an exploded view of an assembly of the two-part vaginal speculum of FIG. 7;

FIG. 9 is a front perspective view of another two-part vaginal speculum, in accordance with another embodiment, having a shaft or insertion portion removable from a handle portion;

FIG. 10 is a front perspective view of the shaft portion of FIG. 9, having a coupling insert extending therefrom for coupling engagement with the handle portion;

FIG. 11 is a perspective view of the coupling insert;

FIG. 12 is a rear elevation view of the shaft portion of FIG. 9, partially cut away to show a placement of the insert or coupler therein;

FIG. 13 is a cross-sectional view of the shaft portion as shown partially cut-away in FIG. 12 along line C-C thereof to further show internal placement of the insert therein;

FIGS. 14 and 15 are opposite exploded perspective views of the handle portion of FIG. 9 showing an assembly thereof; and

FIG. 16 is an exploded perspective view of the speculum of FIG. 9 showing an assembly of the handle portion thereof, as shown in FIGS. 14 and 15, to engage the insert of the shaft portion shown in FIG. 10.

Elements in the several figures are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood elements that are useful or necessary in commercially feasible embodiments are often not depicted in order to facilitate a less obstructed view of these various embodiments of the present disclosure.

### DETAILED DESCRIPTION

Various implementations and aspects of the specification will be described with reference to details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Various apparatuses and processes will be described below to provide examples of implementations of the system disclosed herein. No implementation described below limits any claimed implementation and any claimed implementations may cover processes or apparatuses that differ from those described below. The claimed implementations are not limited to apparatuses or processes having all of the features of any one apparatus or process described below or to features common to multiple or all of the apparatuses or processes described below. It is possible that an apparatus or process described below is not an implementation of any claimed subject matter.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

In this specification, elements may be described as “configured to” perform one or more functions or “configured for” such functions. In general, an element that is configured to perform or configured for performing a function is enabled to perform the function, or is suitable for performing the function, or is adapted to perform the function, or is operable to perform the function, or is otherwise capable of performing the function.

It is understood that for the purpose of this specification, language of “at least one of X, Y, and Z” and “one or more of X, Y and Z” may be construed as X only, Y only, Z only, or any combination of two or more items X, Y, and Z (e.g., XYZ, XY, YZ, ZZ, and the like). Similar logic may be applied for two or more items in any occurrence of “at least one . . . ” and “one or more . . . ” language.

Unless defined otherwise, all technical and scientific terms used herein have the same meaning as commonly understood by one of ordinary skill in the art to which this invention belongs.

Throughout the specification and claims, the following terms take the meanings explicitly associated herein, unless the context clearly dictates otherwise. The phrase “in one of the embodiments” or “in at least one of the various embodiments” as used herein does not necessarily refer to the same embodiment, though it may. Furthermore, the phrase “in another embodiment” or “in some embodiments” as used herein does not necessarily refer to a different embodiment, although it may. Thus, as described below, various embodiments may be readily combined, without departing from the scope or spirit of the innovations disclosed herein.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references unless the context clearly dictates otherwise. The meaning of “in” includes “in” and “on.”

The term “comprising” as used herein will be understood to mean that the list following is non-exhaustive and may or may not include any other additional suitable items, for example one or more further feature(s), component(s) and/or element(s) as appropriate.

The systems and methods described herein provide, in accordance with different embodiments, different examples of a vaginal speculum that overcomes or at least ameliorates one or some of the drawbacks of known products. In general, the embodiments described herein provide a tool that, unlike traditional hinged duck-bill speculums, comprise a solid barrel-type solution for performing cervix visualization and/or various vaginal healthcare procedures such as Pap tests or the like. For instance, the open line-of-sight barrel-type construction of the embodiments described herein allows for direct or instrumented visualization of the cervix (e.g. via associated lighting/camera, to be discussed in further detail below), and/or for the manipulation of swabbing tools therethrough, such as a small, bristled brush to collect cells from inside of the cervix and/or spatula to collect cells from the outside of the cervix, for example. As another example, the open line-of-sight barrel-type construction of the embodiments described herein may allow for direct diagnostic analyses of the cervix, optionally via a camera system or assembly with associated diagnostic software products.

Pap tests and other cervical examinations where traditional speculums are used can cause discomfort and pain to patients. The primary reasons for this discomfort were identified as the process of widening the blades after insertion, temperature and size of the device, and the feeling of resistance upon insertion and removal. This can result in patients avoiding necessary medical care due to fear of the device and the procedure. In addition to the patient's discomfort and pain, practitioners also have issues with traditional devices, which include, but are not limited to, that these devices can only generally be operated with the right hand, that they require additional lighting, and are difficult to maneuver to properly visualize the cervix and vaginal walls. Some practitioners also noted the time it took to warm the device (to make it more comfortable for the patients) and the modifications that needed to be made to complete procedures successfully (such as adding a condom over the speculum to prevent the vagina walls from collapsing into the jaws of the blades). Furthermore, when using a traditional speculum, practitioners must typically maintain contact with the device to retain or support it in place, thus limiting available hands to one to perform any procedure or test necessary. The following description of illustrative embodiments describes various useful alternatives and/or improvements to the traditional speculum.

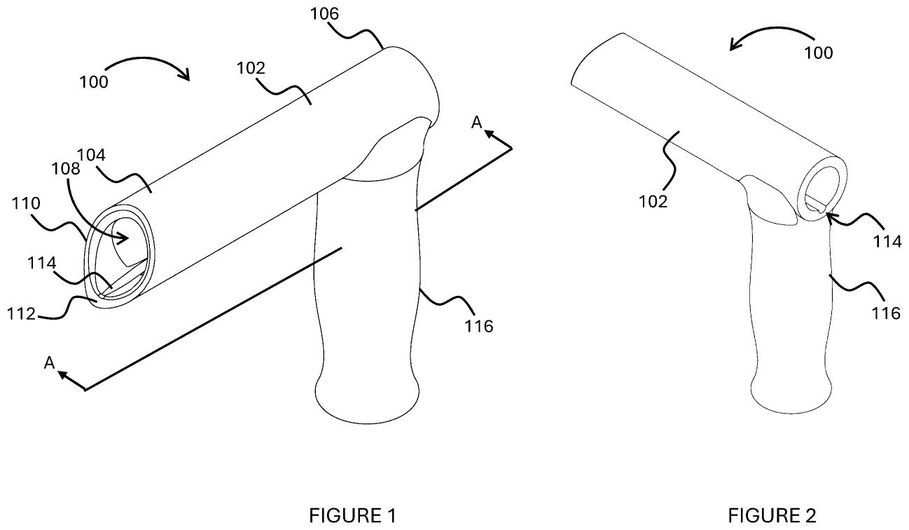

With reference to FIGS. 1 to 6, and in accordance with one exemplary embodiment, a vaginal speculum, generally referred to using the numeral 100, will now be described. In the illustrated embodiment, the speculum 100 generally comprises a shaft or insertion portion 102 having an insertable end 104 that is insertable to widen the vaginal walls of a patient such that a medical practitioner can examine the vaginal canal and/or cervix of the patient (and/or, such that the medical practitioner can deploy tools or instrumentation for such examination), and a distal end 106 opposed thereto from which the device can be handled and/or manipulated during and/or post-insertion. In this example, the shaft portion 102 is generally defined as a tubular insertion portion that defines a barrel-type examination channel 108 extending more or less linearly between the insertable end 104 and distal end 106 and through which medical examination and/or procedures can be performed once the device is inserted, notably providing a line-of-sight channel 108 from the distal end 104 to the insertion end 102.

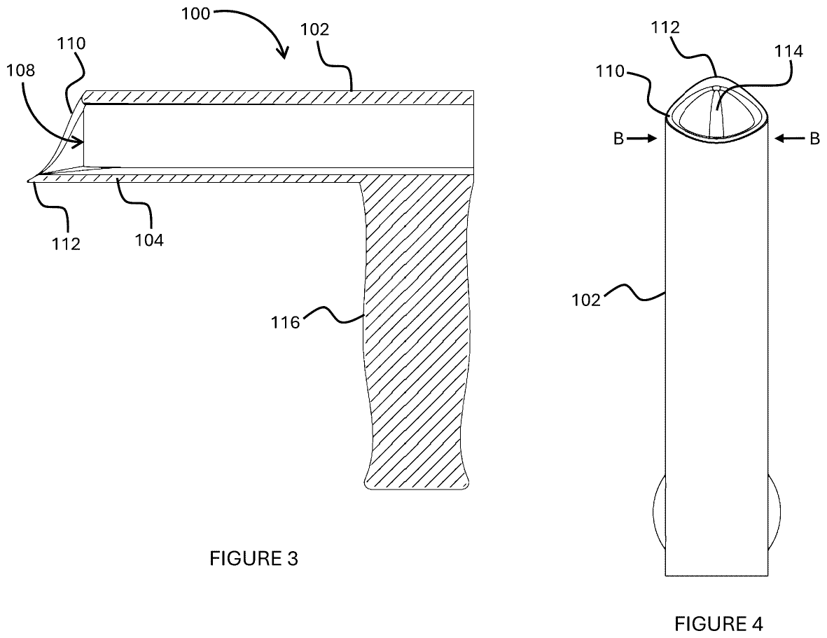

In the illustrated example, the tip of the insertable end 104 is defined by a transversal bevelled cylindrical edge formation 110 that forms an axially extending lip 112 that, in this example, protrudes axially beyond a bevelled transversal plane of the insertable tip. A forward extension of this exemplary lip 112 is best shown in FIGS. 3 and 4, in which the concave forward curving of the bevelled cylindrical edge formation 110 at the tip of the insertable end 104 is more clearly illustrated to terminate in an axially extending lip at an axially utmost edge portion thereof. With specific reference to FIG. 4, at least the tip of the insertable end 104, which extends in this example at least partially from the bevelled cylindrical edge formation 110 and lip 112, is manufactured of a resiliently collapsable material of sufficient flexibility to collapse under manual pressure, for example along arrows B-B, to reduce an insertion cross-section of the resiliently collapsable insertion tip portion for ease of insertion, while being of sufficient resiliency to expand and resist collapse from vaginal wall pressure once inserted. Indeed, in some examples, a significant fraction if not all of the shaft portion 102 may be manufactured of this resiliently collapsable material, though optionally of varying collapsibility, to provide for ease of insertion and comfort during use. For example, in one embodiment, the shaft portion 102 may be manufactured of a medical grade silicone (MSG), or the like, and structured (e.g. shaft wall thickness, resiliency, etc.), at least toward the bevelled edge formation 110 and lip 112, to provide the desired resiliency and collapsibility. For example, the shaft wall thickness of the bevelled edge formation 110 may itself be formed of a progressively decreasing annular thickness so to exhibit a progressively decreasing resiliency and increasing collapsibility toward the very tip of the shaft portion 102.

In other embodiments, the tubular insertion portion 102, or at least the insertable end 104 thereof, comprises an outward bouquet portion or an outward flared portion (not shown) located towards the resiliently collapsable tip portion of the shaft portion 102. The outward bouquet or flared portion is such that the cross-section of the shaft portion 102 varies along its length, having a wider cross-section at the insertable end 104 and a narrower cross-section at the opposed end 106. In some embodiments, the outward bouquet or flared portion provides a use-stabilizing formation. In particular, the outward bouquet or flared portion is configured such that in use, it engages, cups or holds an area proximate a cervix of a patient (optionally, similar to the area that a traditional speculum engages when blades are opened) to increase retention of the vaginal speculum 100 within the patient for the examination or procedure. By comfortably engaging the internal vaginal walls of the patient with the outward bouquet or flared portion, the speculum is held or supported in position, avoiding the need for multiple reinsertions or readjustments, for example, and/or freeing both hands of the practitioner to perform the necessary test or procedure. Additionally, in some embodiments, the outward bouquet or flared portion may improve the field of view of the cervix provided by the disclosed speculum.

In other embodiments, the shaft portion 102 may be manufactured of different materials, for example, where the distal end 106 is more resilient or even generally solid, whereas the insertable end 104 is more resiliently collapsable. In one such example, at least a distal fraction of the insertable portion 102 may be manufactured of a relatively solid and/or rigid material such as stainless steel, plastic, acrylic, or a rigid silicone, and optionally covered or embedded within a MGS or the like, which coating or shell may, in some further examples, monolithically extend to form the resiliently collapsable insertion portion and lip devoid of a solid core/insert material.

In some embodiments, at least a portion of the tubular insertion portion or shaft portion 102 is manufactured of one or more materials selected for transmission of light therethrough. Selection of such material(s) provides increased visibility within the vaginal canal, cervix and/or surrounding tissue, for example. Light transmission both through the walls of the shaft portion 102 towards the vaginal walls, and/or down the walls of the shaft portion 102 towards the cervix, are contemplated. In some embodiments, material(s) selected are optically clear to allow for visualization and/or image capture of the vaginal walls, for example. In some embodiments, transparency is useful, whilst other embodiments with opaque transmission are workable. It is to be appreciated, however, that selection of and/or positioning of optically clear materials is balanced with structural and/or patient comfort considerations related to the shaft portion 102 and/or insertable end 104 thereof. In one embodiment, the shaft portion 102 includes an optically clear window portion arranged along the length of the shaft portion 102 for visualization of the vaginal walls. In some embodiments, at least a portion of the tubular insertion portion or shaft portion 102 includes a surface material or coating of one or more materials selected for transmission of light therethrough, or otherwise a surface finishing selected for light transmission therethrough.

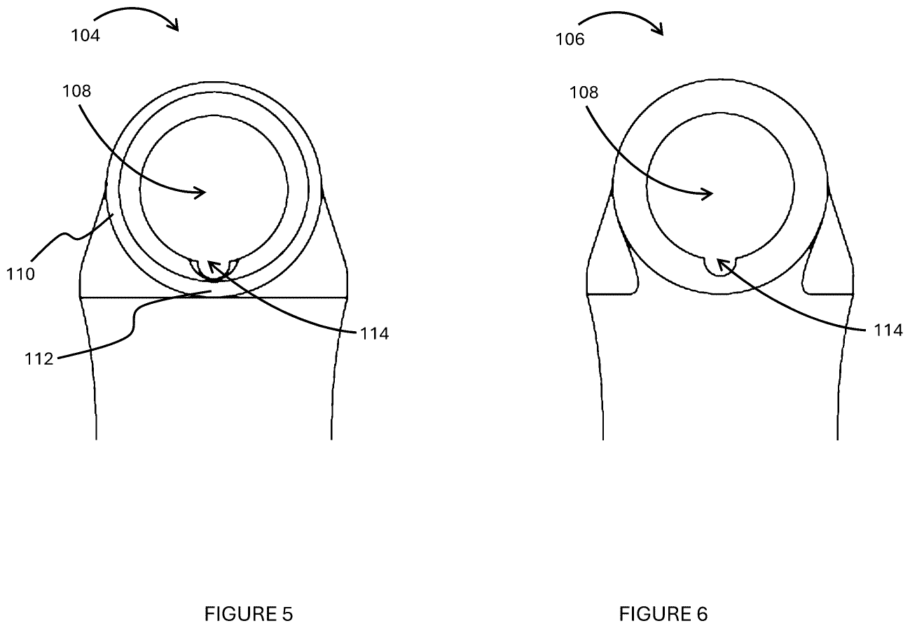

With added reference to FIGS. 5 and 6, in the illustrated embodiment, the inner wall of the shaft portion 102 has formed along a base thereof (i.e. along a base of the generally cylindrical examination channel 108) an axial instrumentation channel or groove 114 shaped and sized to accommodate different medical examination or procedural tools, such as fiber optic illumination and/or imaging tools, to be disposed or secured within the instrumentation channel 114 from the distal end 106 to the insertion end 104, the latter optionally exhibiting additional structural formation feature(s) toward the lip 112 as may be appropriate or required given the instrumentation being employed. In some examples, the instrumentation groove is unobstructively defined within the examination channel, and may further comprise or be defined by a snap-fit groove, securing members, or the like. In some embodiments, the groove may be sealed or integrated within the inner wall of the shaft portion 102 so as to form a channel or conduit which does not expose instrumentation and/or wiring therein to bodily fluids or procedure fluids, for example. In some embodiments, instrumentation cables or fibers may be channeled into the handle portion, for example, for case of management.

With general reference again to FIGS. 1 to 3, in this example, the speculum 100 further comprises a handle portion 116, extending at an angle from the distal end 106 of the shaft portion 102, to facilitate manipulation of the speculum 100 during insertion and/or post-insertion during performance of a medical examination and/or procedure. In this example, the handle portion 116 generally forms a right angle with the shaft portion 102, though other angles may be considered, as will be readily apparent to the skilled artisan. The illustrate handle portion 116 is generally ambidextrous in that it can be easily manipulated with either hand during use.

In this example, the speculum 100 consists of a one-piece design in which the shaft portion 102 and handle portion 116 are integrally formed, for example, of a same material, such as a medical grade silicone (MGS) or the like, or of stainless steel or plastic, for example, optionally covered, at least in part, by a MGS or like material to improve patient comfort, sterilization, durability and/or reusability (and allow for collapsibility at least in the shaft tip region 112). Other examples of material options will be described further below in accordance with different embodiments, to achieve different features, options and/or advantages thereof.

In similar examples, the shaft portion 102 and handle portion 116 may be manufactured separately and fixedly coupled thereafter to provide a similar solution. Alternatively, as will be described in greater detail below, a two-part solution may involve separably engageable shaft and handle portions, for example, where a shaft portion is disposable or more readily replaceable for sterilization, whereas a handle portion is more readily reusable.

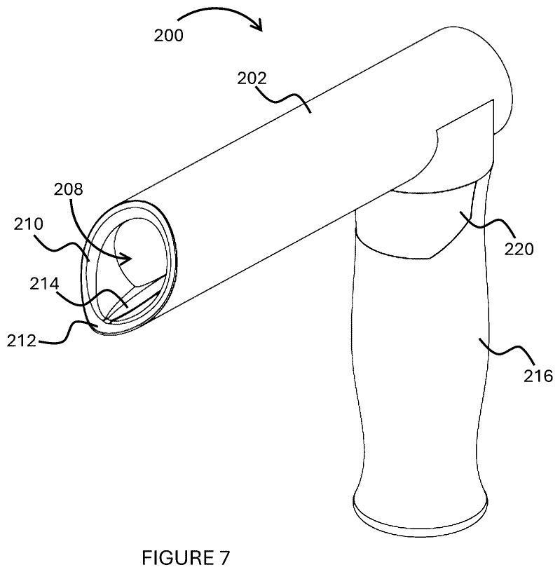

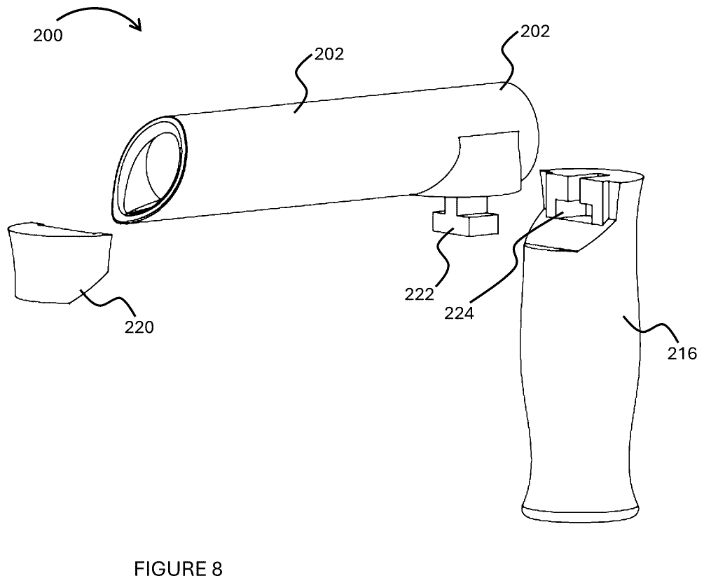

With reference to FIGS. 7 and 8, an alternative speculum, generally referred to using the numeral 200, and in accordance with another embodiment, is illustrated to comprise two parts: a handle portion 216 and a detachably mountable shaft portion 202. The various features and options described above with respect to speculum 100 are equally applicable in respect of speculum 200, in that is also includes a shaft portion 202 manufactured to define a line-of-sight examination channel 208 and a resiliently collapsable shaft tip having a bevelled edge formation 210 and lip 212, and further including an optional instrumentation groove 214 formed along a bottom wall thereof. In this particular embodiment, however, and with particular reference to FIG. 8, the shaft portion 202 further includes a T-shaped coupler 222, integrally formed therewith or structurally assembled thereto at a distal end 206 thereof, that is matingly engageable with a correspondingly T-shaped cavity 224 formed within an upper end of the handle portion 216 so to provide, once assembled, a disengageably fixed junction between the two. A pressure or snap-fitted cover 220 is also provided to encase the junction once set, and removed to permit disengagement of the shaft portion 202 and handle portion 216. Whilst the shaft portion 202 is here described as including the male portion of the coupling, with the handle portion 216 including the female portion, it is to be appreciated that the vice versa configuration is also intended to fall within the scope of the instant disclosure, with the shaft portion 202 including a female coupling formation and the handle portion 216 including a complementary male coupling formation, for example.

As will be appreciated by the skilled artisan, different mechanisms for engaging and disengaging the shaft portion and handle portion may be contemplated, without departing from the general scope and nature of the present disclosure. For example, other coupling mechanisms in other embodiments include a ball-and-socket coupling, a press fit coupling, a friction fit coupling, an interlockable coupling, a spring-loaded coupling, a knuckle joint coupling, a fastener coupling (e.g., bolts, rivets, screws or the like) and/or the like. To provide one non-limiting example, a coupling formation may be provided on the upper end of the handle portion 216, such as a T-shaped coupler similar to 222, with the shaft portion 202 being provided with a complementary cavity, such as a T-shaped cavity similar to 224, without limitation. In one embodiment, the coupling mechanism comprises a silicone cylindrical friction joint, wherein the shaft portion 202 includes a silicone portion or formation which slides over a rigid portion of the handle portion 216 to releasably secure these components together for use. Further, while a rigid or fixed coupling is illustrated herein, alternative coupling mechanisms, such as a hinged or variable angular coupling, may also be considered to provide adjustability in configuration and manipulation of the device.

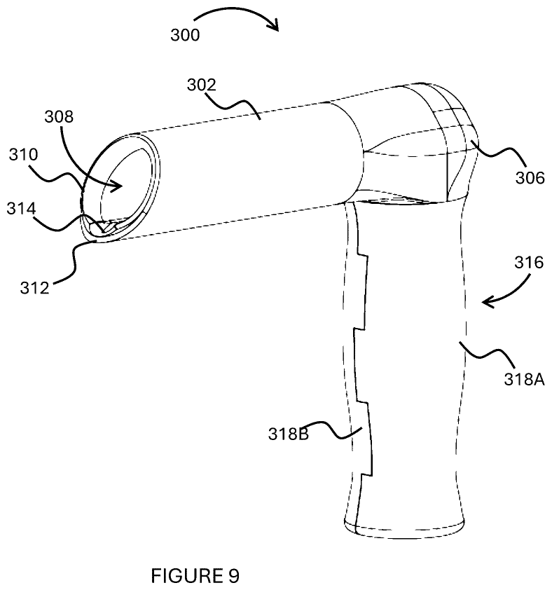

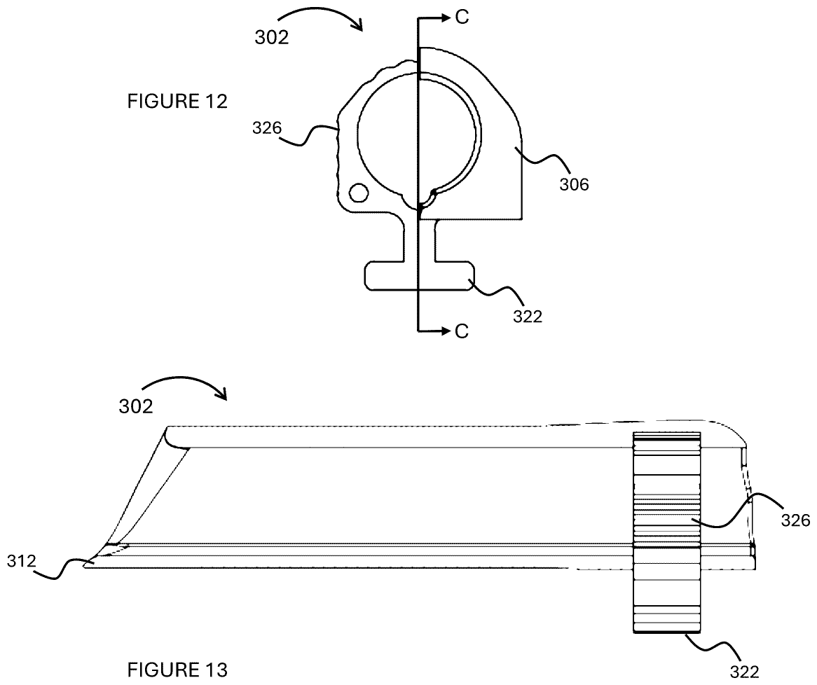

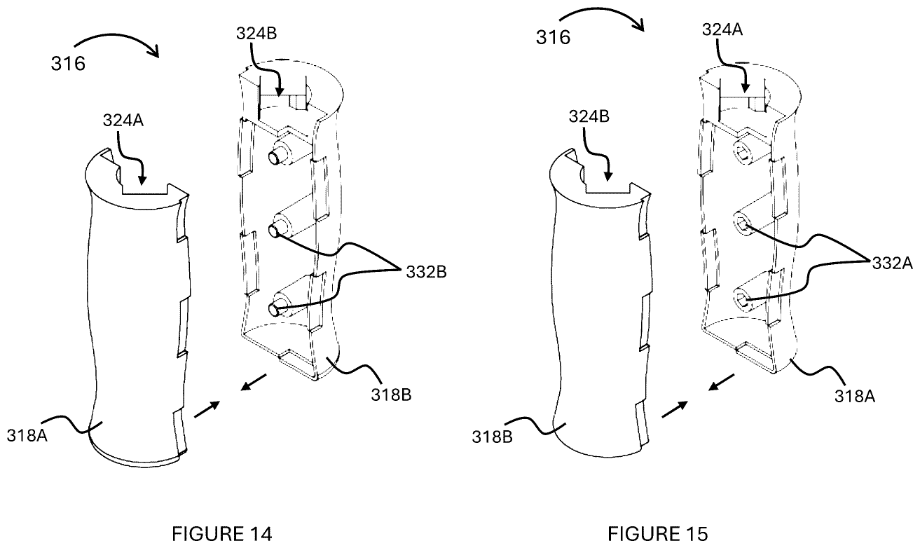

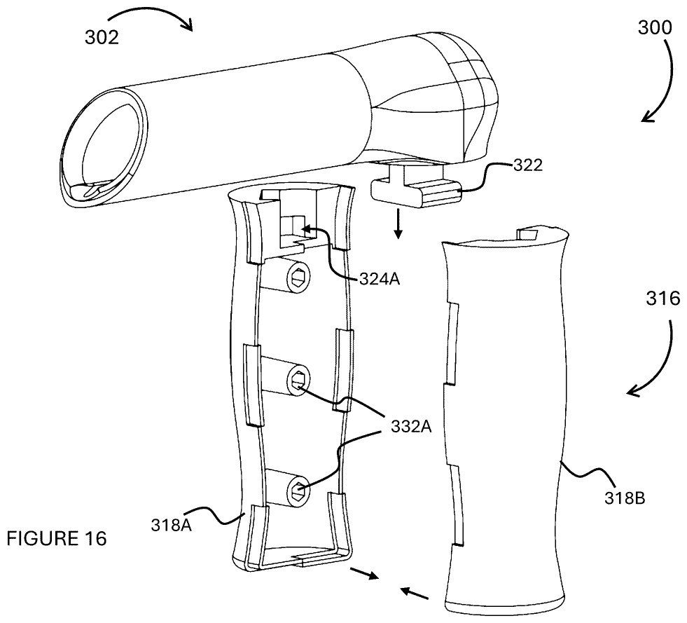

With reference to FIGS. 9 to 16, another embodiment is illustrated of a speculum, generally referred to using the numeral 300, having a distinct two-piece design. In this embodiment, the shaft portion 302 is again disengageably assembled with a handle portion 316 via a T-shaped coupler 322. In this example, however, the T-shaped coupler 322 is engaged within a corresponding T-shaped slot defined by aligning corresponding slot formations 324A and 324B when joining corresponding handle pieces 318A and 318B, respectively. In particular, handle pieces 318A and 318B can be pressure or snap-fitted together by the mating engagement of corresponding couplers 332A and 332B, respectively. This handle design allows for a simple and easy assembly of the speculum 300, and can be manufactured of various materials, such as stainless steel, plastic, acrylic, or a rigid silicone, as may be the case and without limitation.

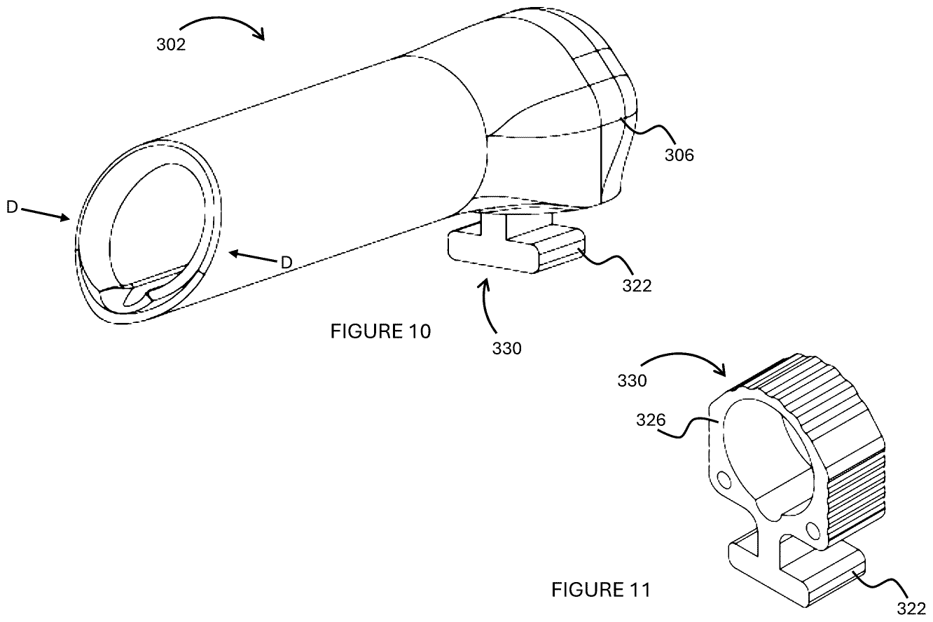

With particular reference to FIGS. 10 to 12, the T-shaped coupler 322 in this examples forms part of a solid insert 330, which can be manufactured of stainless steel or the like in some examples, that comprises a ring-shaped component that can be molded or otherwise inserted within the distal end 306 of the shaft portion 302, such that the ring-shaped component circumscribes the examination channel 308 while providing the T-shaped coupler 322 to extend outwardly therefrom for mechanical coupling with the handle portion 316. For example, in one embodiment, a stainless-steel insert 330 may be embedded within the shaft portion during formation thereof, for example, when otherwise entirely manufactured as a resiliently collapsable MGS tube, or the like.

As with previous examples, the shaft portion 302 is manufactured to define a line-of-sight examination channel 308 and a resiliently collapsable shaft tip having a bevelled edge formation 310 and lip 312 that is resiliently collapsable, for example, under manual pressure, along arrows D-D of FIG. 10, to facilitate insertion and improve patient comfort, for example. An optional instrumentation groove 314 is again formed along a bottom wall of the examination channel 308, as described above.

In the instant disclosure, where reference is made to the tip being resiliently collapsible under “manual” pressure, it is to be appreciated that such resiliently collapsibility is imparted by the shape, configuration, orientation and/or material(s) selection, in different embodiments. Examples are apparent from the embodiments disclosed herein. Additionally, in some embodiments, the tip or insertable end 104 of the shaft portion 102 includes one or more formations to assist or impart resilient collapsibility or foldability. For example, one embodiment envisaged includes ridge and groove collapse formations located towards or at the tip which operably gather to aid collapse or folding of the tip for insertion. Other folding, buckling or deformation lines, formations or structures are envisaged for other embodiments.

As noted above, the embodiments of the vaginal speculum disclosed herein provide or enable the usage of one or more technology components in association therewith. Such technology components serve various purposes, including improving visualization, assisting in data capture, diagnostics, or the like, or indeed a combination thereof. In one embodiment, the vaginal speculum is configured to accommodate a camera system, which includes lighting elements. In this embodiment, the camera system fits compactly within the groove formed in the examination channel, thereby to avoid any obstruction of the examination channel. In different embodiments, the camera system is deployable in different modes, including a continuous capture mode, where continued image data for a single examination is captured as a video, for example, or a discrete capture mode, where a series of single images representative of an examination are captured, for example. It is to be appreciated that different modes may be suited to different patient presentations. Typically, the camera and lighting system envisaged is operably coupled to a digital data memory for storing the data acquired, which in turn is accessible by a software product running on a user device (e.g., personal computer, iPad, mobile phone or the like), configured to process the data stored. Notwithstanding same, some embodiments include immediate data processing at data capture. In one embodiment, the software product is configured to execute stored instructions to associate data captured/stored with a particular patient data record, optionally in conjunction with practitioner examination inputs (e.g., a diagnosis, medical recommendations provided, and/or the like). In one embodiment, the software product is configured to execute stored instructions to perform diagnostic analyses on the data captured/stored. For example, if a patient medical record has been established, including at least one prior image of a patient's cervix, comparison of a current image of the patient's cervix to the at least one prior image, using a diagnostic image-based rule set, can yield a potential diagnosis or lack thereof, to provide but one non-limiting example. Indeed, it is to be appreciated that image capture provided by the camera system may allow the practitioner to perform, or otherwise a software product to perform, diagnostic monitoring of the cervix over time.

In some embodiments, the handle portion 216 has integrated therein structures and/or formations to retain therein electrical wiring and/or componentry associate with the camera and/or lighting system. For example, the handle portion 216 may include a universal serial bus (USB) port for transfer of images from the speculum to an external storage medium, in one embodiment. Whilst wired connectivity is envisaged in some embodiments, other embodiments rely on wireless communication of data acquired to an external storage medium, such as via a short-range wireless communication protocol like Bluetooth®, WiFi® or the like. As another example, the handle portion 216 may include a power port for electrically powering the camera and/or lighting system, in one embodiment.

In some embodiments, the coupling between the handle portion 216 and the shaft portion 202 provides one or more channels, recesses or formations configured to retain therein electrical wiring and/or or componentry associated with the camera and/or lighting system. In two-piece embodiments, where the handle portion 216 and shaft portion 202 are separable, such formations are arranged to align when the speculum is assembled.

Whilst specific shapes and dimensions are illustrated as non-limiting examples herein, it is to be appreciated that various shaft 102 and tip designs, shapes and sizes are envisaged in different embodiments, and are excluded here merely for the sake of brevity. For example, variations in shapes and dimensions may be required to adapt the disclosed speculum for different procedures and anatomies, all of which are intended to fall within the scope of the instant disclosure.

Turning now to another aspect of the disclosure, there is provided a vaginal examination kit (not shown). The kit generally comprises a handle portion; and at least two exchangeable tubular insertion portions disengageably mountable at an angle to the handle portion at a manipulatable end thereof, and defining a line-of-sight channel therein from the manipulatable end to a resiliently collapsable insertion tip portion of the tubular insertion portion. For each tubular insertion portion, the resiliently collapsable insertion tip portion is manufactured of a resiliently collapsable material of sufficient flexibility to collapse under manual pressure to reduce an insertion cross-section thereof for ease of insertion, while being of sufficient resiliency to expand and resist collapse from vaginal wall pressure once inserted.

In some embodiments, the tubular insertion portions are provided with differing shapes, dimensions and/or configurations, optionally to accommodate different anatomies, examinations and/or procedures. For example, a tubular insertion portion with a narrower maximum circumference may be advantageous in some use cases, whereas a wider maximum circumference may be advantageous in other use cases.

In some embodiments, the handle portion is manufactured to be reusable, optionally being configured to withstand sterilization between uses (e.g., via autoclave and/or chemical sterilization). Selection of a material(s) suited for multiple, potentially oxidising sterilization, is thus requisite for the handle portion in such embodiments.

In some embodiments, the tubular insertion portions are manufactured to be single-use disposables. To this end, the kit may include a plurality of insertion portions in some embodiments, optionally of various shapes, dimensions and/or configurations, to accommodate different anatomies, examinations or procedures, such that a medical practitioner can select the tubular insertion portion best suited to the use case. Notably, selection of material(s) for the tubular insertion portion, when disposable, may consider manufacturing cost of such disposables, and optionally, recycling options, to provide a few examples of material selection considerations.

The handle portion and tubular insertion portions include a coupling means, which allows the interchangeable coupling of each tubular insertion portion to the handle. For example, each tubular insertion portion comprises a coupler extending radially therefrom, in some embodiments, allowing for engagement and disengagement of the tubular insertion portions with the handle portion.

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Except to the extent necessary or inherent in the processes themselves, no particular order to steps or stages of methods or processes described in this disclosure is intended or implied. In many cases the order of process steps may be varied without changing the purpose, effect, or import of the methods described.

Information as herein shown and described in detail is fully capable of attaining the above-described object of the present disclosure, the presently preferred embodiment of the present disclosure, and is, thus, representative of the subject matter which is broadly contemplated by the present disclosure. The scope of the present disclosure fully encompasses other embodiments which may become apparent to those skilled in the art, and is to be limited, accordingly, by nothing other than the appended claims, wherein any reference to an element being made in the singular is not intended to mean “one and only one” unless explicitly so stated, but rather “one or more.” All structural and functional equivalents to the elements of the above-described preferred embodiment and additional embodiments as regarded by those of ordinary skill in the art are intended to be encompassed by the present claims. Moreover, no requirement exists for a system or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, as may be apparent to those of ordinary skill in the art, are also encompassed by the disclosure.

What is claimed is:

1. A vaginal speculum, comprising
a handle portion; and
a tubular insertion portion mounted at an angle to said handle portion at a manipulatable end thereof, and defining a line-of-sight channel therein from said manipulatable end to a resiliently collapsable insertion tip portion of the tubular insertion portion;
wherein said resiliently collapsable insertion tip portion is manufactured of a resiliently collapsable material of sufficient flexibility to collapse under manual pressure to reduce an insertion cross-section thereof for ease of insertion, while being of sufficient resiliency to expand and resist collapse from vaginal wall pressure once inserted.

2. The vaginal speculum of claim 1, wherein said resiliently collapsable tip portion comprises a bevelled insertion edge formation.

3. The vaginal speculum of claim 2, wherein said bevelled insertion edge formation further comprises an axially extending lip at an axially utmost edge portion thereof.

4. The vaginal speculum of claim 3, wherein said beveled insertion edge formation is defined by a concave forward curving edge terminating in said axially extending lip.

5. The vaginal speculum of claim 1, wherein said resiliently collapsable tip portion is defined by a decreasing annular thickness.

6. The vaginal speculum of claim 1, wherein said tubular insertion portion comprises an outward bouquet portion located towards said resiliently collapsable tip portion.

7. The vaginal speculum of claim 1, wherein said tubular insertion portion is integrally formed, with said resiliently collapsable tip portion, of said resiliently collapsable material.

8. The vaginal speculum of claim 7, wherein a collapsibility of said resiliently collapsable tip portion is greater than that of said manipulable end.

9. The vaginal speculum of claim 1, wherein said resiliently collapsable tip portion comprises a manually collapsible formation to facilitate resilient collapse.

10. The vaginal speculum of claim 9, wherein said manually collapsible formation comprises a plurality of grooves running on any one or both of an interior surface or an exterior surface of said tubular insertion portion.

11. The vaginal speculum of claim 1, wherein said resiliently collapsable material comprises any one or combination of: a medical grade silicone, a polysiloxane, an elastomer, or a biocompatible flexible resin.

12. The vaginal speculum of claim 11, wherein said tubular insertion portion is manufactured of any one of stainless steel, plastic, acrylic, or a rigid silicone; and overlayed by any one or combination of medical grade silicone, polysiloxane, elastomer or a biocompatible flexible resin, to form said resiliently collapsable insertion tip portion extending axially therefrom.

13. The vaginal speculum of claim 1, wherein at least a portion of said tubular insertion portion is manufactured of one or more materials selected for transmission of light therethrough.

14. The vaginal speculum of claim 1, wherein said tubular insertion portion further comprises a coupler extending radially therefrom for coupling to said handle portion, and wherein said coupler comprises a disengageable coupling portion allowing for engagement and disengagement of said tubular insertion portion with said handle portion.

15. The vaginal speculum of claim 1, wherein said tubular insertion portion and said handle portion are mechanically coupled via a hinge mechanism to selectively adjust said angle.

16. The vaginal speculum of claim 1, wherein an unobstructive instrumentation groove is defined along an internal surface of said tubular insertion portion, or within an annular wall of said tubular insertion portion, to receive instrumentation therein for performing a medical examination or procedure.

17. The vaginal speculum of claim 16, wherein said groove further comprises one or more securing members along a length thereof to secure said instrumentation therein.

18. The vaginal speculum of claim 16, wherein said groove is dimensioned to securely receive therein any one or both of: a lighting assembly or a camera assembly, to channel any one or both to within said handle portion.

19. A vaginal examination kit, comprising:
a handle portion; and
at least two exchangeable tubular insertion portions disengageably mountable at an angle to said handle portion at a manipulatable end thereof, and defining a line-of-sight channel therein from said manipulatable end to a resiliently collapsable insertion tip portion of the tubular insertion portion;
wherein said resiliently collapsable insertion tip portion is manufactured of a resiliently collapsable material of sufficient flexibility to collapse under manual pressure to reduce an insertion cross-section thereof for ease of insertion, while being of sufficient resiliency to expand and resist collapse from vaginal wall pressure once inserted.

20. The vaginal examination kit of claim 19, wherein each of said tubular insertion portions comprise a coupler extending radially therefrom allowing for engagement and disengagement of said tubular insertion portions with said handle portion, and wherein each of said tubular insertion portions are single-use disposables.
